(19) United States
(12) Patent Application Publication
(10) Pub. No.: US 2003/0024528 A1
(43) Pub. Date: Feb. 6, 2003

(54) Moisture trap

(76) Inventor: James E. Graham, Swampscott, MA (US)

Correspondence Address: Iandiorio & Teska, 260 Bear Hill Road, Waltham, MA, 02451-1018, US

(21) Appl. No.: 10/174,680

(22) Filed: Jun. 19, 2002

Related U.S. Application Data
(60) Provisional application No. 60/310,054, filed on Aug. 4, 2001.

(51) Int. Cl. A61M 15/00

(52) U.S. Cl. 128/201.13; 128/200.24

(57) ABSTRACT

A moisture trap including a housing with an inlet and an outlet and defining a reservoir therein. A conduit in the housing extends through the reservoir from the inlet and the conduit has a distal open end spaced from the housing outlet. A hydrophilic filter canister is received in the housing filling the reservoir. The canister includes an end wall disposed over the distal open end of the conduit, the end wall having gas ports therethrough in fluid communication between the housing outlet and the distal open end of the conduit. A hydrophilic column extends from the end wall within the conduit for collecting liquid and wicking collected liquid into the reservoir. A hydrophobic filter is disposed between the hydrophilic filter canister and the housing outlet to block liquid from passing through the outlet.

### RELATED APPLICATIONS

This application claims priority of U.S. Provisional application Serial No. 60/310,054, filed Aug. 4, 2001.

### FIELD OF THE INVENTION

This invention relates to a moisture trap for removing moisture from patient exhalation.

### BACKGROUND OF THE INVENTION

In the course of some medical procedures, it is often desirable to monitor a patient's exhalation and, in some instances, its gaseous composition for detecting apnea conditions or analyzing the exhalation of a patient under anesthesia. Normally, a collection device in the form of a mask is placed over the patient's nose and mouth and a conduit connects the mask to a sensing device typically incorporating infrared spectrometer technology.

Accurate analysis of the gases in a patient's exhalation depends on the collection of the flow of exhalation without the introduction of factors which might distort the results of the analysis. Introduction of contaminants into the flow, or other alteration of the exhalation in the monitoring system can render analytical results which do not reflect the actual condition of the patient.

The patient's exhalation is usually relatively humid and moisture can condense as the exhalation flows from the patient to the sensor and this moisture must be removed before the exhalation reaches the sensor. Otherwise, condensation can result in inaccurate readings at the sensor and condensation can interrupt the smooth flow of exhalation to the sensor distorting the operation of the sensor. Also, infrared spectrometer sensing devices typically used in patient monitoring systems are often delicate and can be adversely effected by moisture entering the sensor.

It is thus known to place a moisture trap between the patient and the sensing device to separate moisture from the exhalation before it enters the sensing device. Some prior art moisture traps utilize porous hydrophilic material to separate water vapor from the flow of exhalation. While hydrophilic materials can effectively remove a quantity of condensed moisture from a flow of humid gas, their use in some moisture trap designs have introduced other problems. Hydrophilic materials are porous and include many voids and, in the prior art, moisture is typically removed from the exhalation by allowing the exhalation to pass in proximity with or through the hydrophilic material. Gases, however, enter the hydrophilic material and then later become re-entrained with and mix with the flow of the exhalation resulting in erroneous readings. The greater the volume of hydrophilic material exposed to the flow of exhalation, generally the greater the volume of gas which can be stored therein and is then be available to later contaminate the exhalation. Minimizing the volume of hydrophilic material used in some prior art designs can decrease the amount of such mixing, but it can also undesirably decrease the capacity of the moisture trap. See U.S. Pat. No. 5,131,387 incorporated herein by this reference.

Moreover, typical moisture traps with hydrophilic or hydrophobic materials therein are often orientation sensitive in that water collects in a bottom chamber. Such traps also exhibit low capacities, and/or include large dead spaces which distort sensor readings. One neonatal moisture trap includes a number of U-shaped tubes designed to trap moisture but this device is complex and high cost (e.g., $3.00) and, moreover, has an extremely low capacity.

The '387 patent discloses a moisture trap with a central passageway between an inlet and an outlet with only a single restricted opening into an adjacent reservoir filled with hydrophilic material. The central passageway includes a column of hydrophilic material and the restricted opening in the passageway is completely filled with a hydrophilic wick contacting on one end the column of hydrophilic material and on the other end the hydrophilic material in the reservoir. The wick thus fills with water and prevents exhalation gases from escaping into the reservoir.

This device is commercially successful but difficult to manufacture and fairly high in cost (e.g. $10.00). Worse, this device, because of its size, has an inordinate amount of dead space rendering it unsuitable for neonatal uses where the breath rate can be as high as 150 breaths per minute. And, were this device made smaller, the manufacturing problems associated with forming a central passageway with a single restrictive opening therein filled with a hydrophilic wick would decrease the reliability of the product and increase the manufacturing problems associated with it.

### SUMMARY OF THE INVENTION

It is therefore an object of this invention to provide a different and improved moisture trap.

It is a further object of this invention to provide such a moisture trap which does not suffer from reliability or manufacturability problems because there is no single restrictive opening.

It is a further object of this invention to provide such a moisture trap which is not position sensitive.

It is a further object of this invention to provide such a moisture trap which has a larger capacity.

It is a further object of this invention to provide such a moisture trap which has a small amount of dead space.

It is a further object of this invention to provide such a moisture trap which can be manufactured and sold at a low price.

It is a further object of this invention to provide such a moisture trap which, in one embodiment, is specifically suited to neonatal uses.

It is a further object of this invention to provide such a moisture trap which is easy to manufacture.

It is a further object of this invention to provide such a moisture trap which does not suffer from reliability problems.

The invention results from the realization that a simple to construct and assemble moisture trap useful in neonatal applications is effected, in the preferred embodiment, by a two-section housing and a unique integrated hydrophilic canister disposed between the two housing sections.

This invention features a moisture trap preferably comprising a housing including an inlet and an outlet and defining a reservoir therein. A conduit in the housing extends through the reservoir from the inlet and the conduit has a distal open end spaced from the housing outlet. A hydrophilic filter canister is received in the housing filling the reservoir. The canister includes an end wall disposed over the distal open end of the conduit and the end wall has gas ports therethrough in fluid communication between the housing outlet and the distal open end of the conduit. A hydrophilic column extends from the end wall within the conduit for collecting liquid and wicking the collected liquid into the reservoir. Also typically included is a hydrophobic filter disposed between the hydrophilic filter canister and the housing outlet to block liquid from passing through the outlet. Typically, the conduit is funnel shaped and increases in diameter at the distal end thereof.

The canister is preferably a cylindrical body having a centrally extending channel therein which receives the conduit and the housing is typically less than 1¼ inch in diameter and less than 1¼ inch in length. The conduit is approximately 2 mm in diameter.

The housing may comprise a plurality of sections such as a first section including an end plate comprising the inlet and the conduit. The second section includes an end plate comprising the outlet and an outer wall extending from the end plate.

A moisture trap for neonatal uses in accordance with this invention preferably features a housing less than 1¼ inch in diameter and less than 1¼ inch in length and including an inlet and an outlet and defining a reservoir therein, a conduit approximately 2 mm in diameter in the housing extending through the reservoir from the inlet, the conduit having a distal open end spaced from the housing outlet, and a hydrophilic filter canister in the shape of a cylindrical body with a centrally extending channel therein which receives the conduit of the housing. The canister has an end wall disposed over the distal open end of the conduit, the end wall having gas ports therethrough in fluid communication between the housing outlet and distal open end of the conduit, and a hydrophilic column extending from the end wall within the conduit and surrounded by the channel for collecting liquid and wicking collected liquid into the reservoir. A hydrophobic filter is typically disposed between the hydrophilic filter canister and the housing to block liquid from passing through the outlet.

One moisture trap in accordance with this invention features a housing including an inlet and an outlet and defining a reservoir therein, a conduit in the housing extending through the reservoir from the inlet, the conduit having a distal open end spaced from the housing outlet, hydrophilic material received in the housing filling the reservoir, and hydrophilic material disposed over the distal open end of the conduit with gas ports therethrough in communication between the housing outlet and the distal open end of the conduit.

Preferably a hydrophilic column extends from the hydrophilic material disposed over the distal open end of the conduit and within the column for collecting liquid and wicking collected liquid into the reservoir. Also, a hydrophobic filter is preferably disposed between the hydrophilic material disposed over the distal open end of the conduit and the housing outlet to block liquid from passing through the outlet.

The hydrophilic material disposed over the distal open end of the conduit and the hydrophilic material received in the housing filling the reservoir is preferably in the form of a single integral canister with a hydrophilic column received in the conduit.

### BRIEF DESCRIPTION OF THE DRAWINGS

Other objects, features and advantages will occur to those skilled in the art from the following description of a preferred embodiment and the accompanying drawings, in which:

FIG. 1 is a schematic view showing a complete neonatal exhalation sensing system including a moisture trap;

FIG. 2 is a schematic partially cut-away view of one typical prior art moisture trap;

FIG. 3 is a schematic view showing the U-shaped tubes of a prior art neonatal moisture trap;

FIG. 4 is a schematic cross-sectional view showing the primary components associated with the moisture trap of U.S. Pat. No. 5,131,387;

FIG. 5 is a schematic cross-sectional view showing the primary components associated with the preferred embodiment of the moisture trap of this invention;

FIG. 6 is a schematic exploded top view of the moisture trap shown in FIG. 5;

FIG. 7 is a schematic exploded bottom view of the inventive moisture trap shown in FIG. 5; and

FIG. 8 is a schematic cross-sectional view showing the unique hydrophilic canister of the subject invention.

### DISCLOSURE OF THE PREFERRED EMBODIMENT

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings.

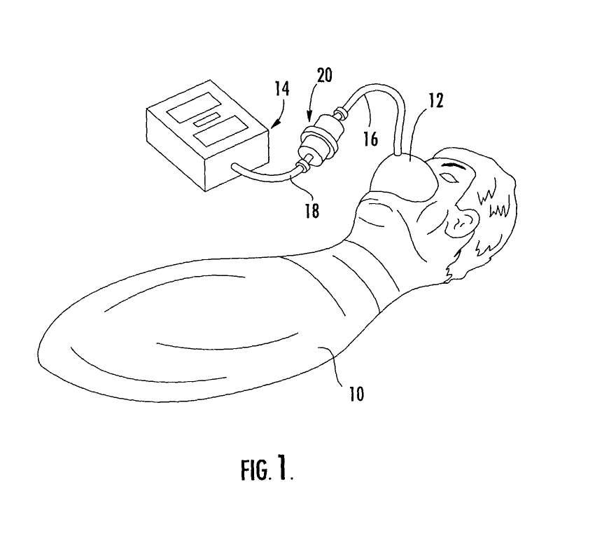

This invention relates to a moisture trap primarily but not exclusively useful in neonatal applications as shown in FIG. 1 wherein infant 10 is fitted with device 12 for collecting a flow of exhalation. In one example, collection device 12 is a mask placed over the infant's nose and mouth, but other standard collection devices can be used as well. Sensing device 14, chosen from various configurations including conventional infrared spectrometers, is operable to analyze the gaseous composition of the infant's exhalation including, for example, carbon-dioxide or anesthetic gas content. Sensing device 14 may also include an apnea monitor (not shown) for detecting a complete stoppage of the flow of exhalation.

A pair of conduits 16 and 18 communicate between collection device 12 and sensing device 14 to provide the flow path for the exhalation. Conduits 16 and 18 can be standard conduits or cannulas. In the preferred embodiment, the conduits are made of a conventional flexible clear plastic. Moisture trap 20 is connected between conduits 16 and 18 and thus located between collection device 12 and sensing device 14. Trap 20 is provided to remove moisture from the flow of exhalation to protect sensing device 14 from the introduction of moisture and to ensure the reliability of the sensor as a patient monitor.

As stated in the Background section above, trap 20 must not alter the gaseous composition of the exhalation being monitored by sensing device 14. Also, a preferred sensing device should be low in cost, easy to manufacture, reliable, position insensitive, of high capacity, and include minimal dead space. Unfortunately, the prior art does not teach or suggest a device which meets all of these goals.

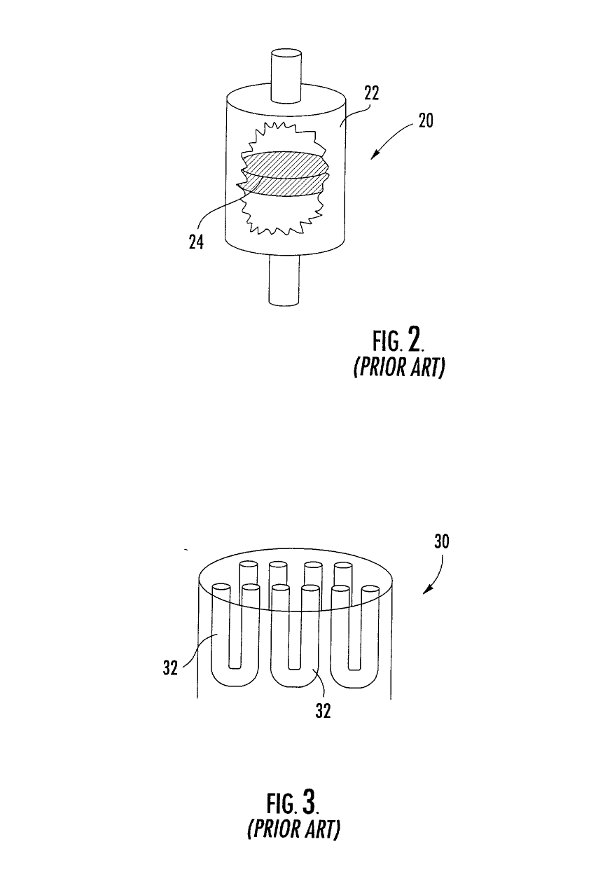

For example, prior art moisture trap 20, FIG. 2 includes housing 22 with hydrophilic material 24 therein. As delineated in the Background section above and in U.S. Pat. No. 5,131,387, such devices cause distortion when the gaseous composition of the exhalation being monitored is held in the porous hydrophilic material and then becomes re-entrained and mixes with the flow of exhalation. Moisture trap 20 is also position sensitive in that water collects in the lower half thereof and thus moisture trap 20 must be continuously maintained in the orientation shown in FIG. 2.

Prior art moisture trap 30, FIG. 3 is specifically designed for neonatal uses where the breath rate can be as high as 150 breaths per minute. U-shaped tubes 32 trap moisture but this device is difficult to manufacture and thus is fairly high in price and also very low in capacity holding only a few drops of water.

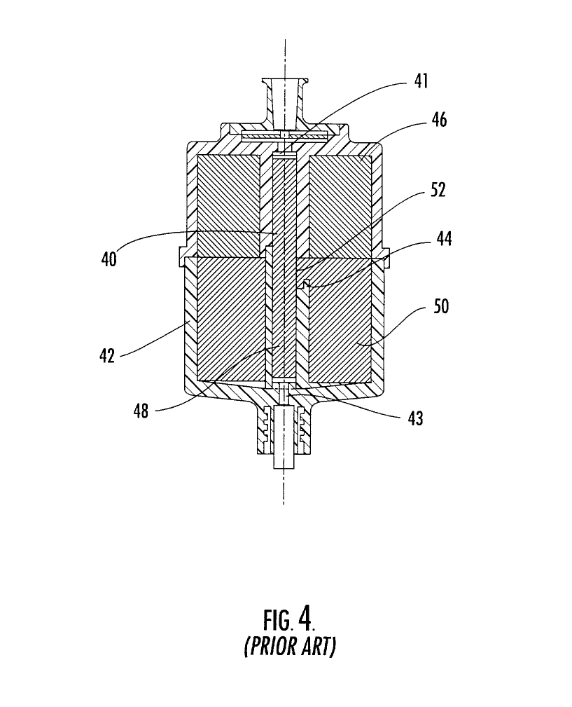

A schematic representation of the prior art moisture trap disclosed in the '387 patent is shown in FIG. 4. Central passageway 40 in housing 42 between apertures 41 and 43 includes single restricted opening 44 in communication with reservoir 46. Hydrophilic column 48 extends in central passageway 40, hydrophilic material 50 fills reservoir 42, and hydrophilic wick 52 fills restricted opening 44. Exhalation gases are prevented from entering reservoir 46 to reduce distortion of the gas content when wick 52 fills with water and plugs single restricted orifice 44. Water, in turn, is collected by hydrophilic column 48 and urged into hydrophilic material 50 in reservoir 46 by wick 52.

Although this device is commercially successful, it is difficult and costly to manufacture central passageway 40 with only a single restricted opening 44 filled with wick 52. Moreover, because of its size, this device cannot be used in neonatal applications and, were it made smaller, the reliability and manufacturability problems associated with it would only increase.

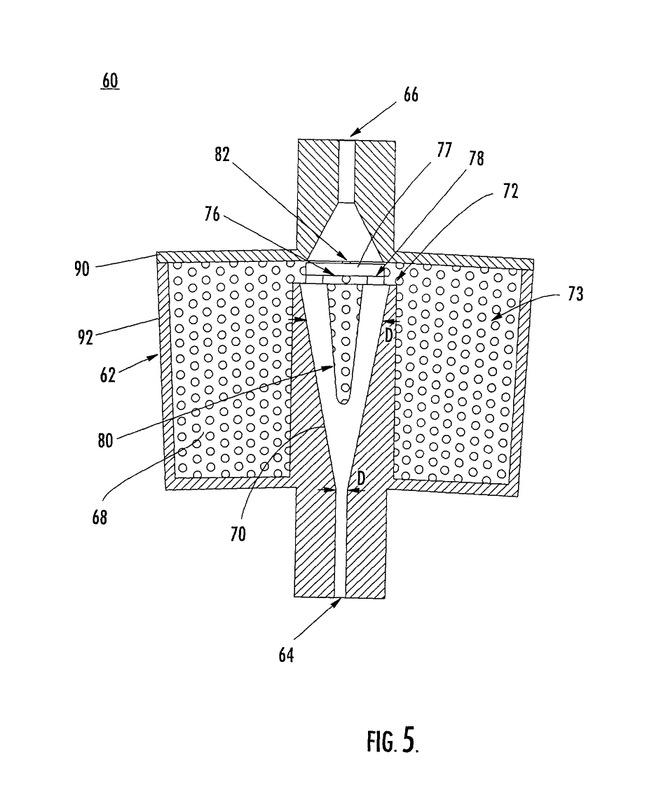

In this invention, in contrast, trap 60, FIG. 5 does not have a central passageway with a single restricted opening and thus the manufacturability and reliability problems associated therewith are significantly reduced to the point where housing 62 can be made small enough (e.g., ¾ of an inch long by ¾ of an inch in diameter) for neonatal uses where the breath rate is as high as 150 breaths per minute.

Housing 62 includes inlet 64 and outlet 66 which may include structure forming luer connections as known in the art. Housing 62 defines reservoir 68 and conduit wall 70 in housing 62 extends from inlet 64 continuously as shown through reservoir 68 but has distal end 72 spaced from housing outlet 66. As shown, conduit wall 70 is preferably funnel shaped with narrow diameter d and wider diameter D.

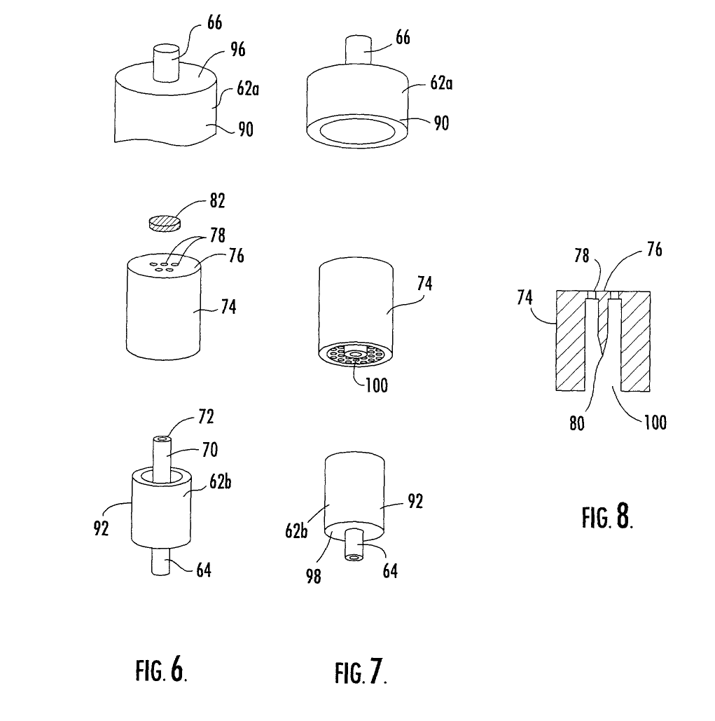

Hydrophilic material 73 preferably in the form of canister 74 (see FIGS. 6-8) is received in housing 62 and hydrophilic material 73 thus fills reservoir 68. Canister 74 includes hydrophilic material end wall 76 disposed over the distal open end 72 of conduit 70. End wall 76, in turn, has gas ports 78 therethrough in fluid communication between outlet 66 of housing 62 and conduit 70 for passing exhalation gases therebetween. Item 77 is an optional airspace. Also, preferably, canister 74 includes hydrophilic column 80 extending from end wall 76 within conduit 70 for collecting liquid and wicking the collected liquid by virtue of end wall portion 76 into reservoir 68 filled with hydrophilic material 73. Also shown is hydrophobic filter 82 disposed in outlet 66 adjacent end wall 76 of canister 74.

FIGS. 6-7 depict the simplicity of manufacturing the preferred embodiment of the moisture trap of this invention. The housing, typically less than 1¼ inch in diameter and less than 1¼ inch in length, is formed in two sections: first section 90 and second section 92 sonically welded together. First section 90 includes end plate 96 and inlet 66 therein with outer wall 62a extending therefrom. Second section 92 includes end plate 98 with outer wall 62b extending therefrom and inlet 64 therethrough. Second section 92 also includes conduit 70 (e.g., 2 mm in diameter) with distal open end 72 as shown. Hydrophilic canister 74 is typically a cylindrical body as shown with centrally extending channel 100 therein surrounding hydrophilic column 80, FIGS. 5 and 8. End wall 76, FIG. 6 of canister 74 includes gas ports 78 typically covered by hydrophobic filter 82.

In this way, moisture trap 60, FIGS. 5-7 in combination with hydrophilic canister 74 is simple to construct and assemble and does not suffer from reliability or manufacturability problems because there is no single restricted central passageway opening. The water filter or moisture trap design of the subject invention is low in cost (e.g. $3.00), easier to manufacture, more reliable, not position sensitive, has a higher capacity with a minimum of dead space, and can be specifically constructed for neonatal uses although other uses, configurations, and sizes are possible.

Thus, although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

Other embodiments will occur to those skilled in the art and are within the following claims:

What is claimed is:

1. A moisture trap comprising:
a housing including an inlet and an outlet and defining a reservoir therein;
a conduit in the housing extending through the reservoir from the inlet, the conduit having a distal end spaced from the housing outlet;
a hydrophilic filter canister received in the housing filling the reservoir and including:
an end wall disposed over the distal open end of the conduit, the end wall having gas ports therethrough in fluid communication between the housing outlet and the distal open end of the conduit, and
a hydrophilic column extending from the end wall within the conduit for collecting liquid and wicking said liquid into the reservoir; and
a hydrophobic filter disposed between the hydrophilic filter canister and the housing outlet to block liquid from passing through the outlet.

2. The moisture trap of claim 1 in which the canister is a cylindrical body having a centrally extending channel therein which receives the conduit.

3. The moisture trap of claim 1 in which the housing is less than 1¼ inch in diameter and less than 1¼ inch in length.

4. The moisture trap of claim 1 in which the conduit is approximately 2 mm in diameter.

5. The moisture trap of claim I in which the housing comprises a plurality of sections.

6. The moisture trap of claim 5 in which the housing includes two sections, a first section including an end plate comprising the inlet and the conduit; and a second section including an end plate comprising the outlet, and an outer wall extending from the end plate.

7. The moisture trap of claim 1 in which the conduit is funnel shaped and increases in diameter at the distal end thereof.

8. A moisture trap for neonatal uses comprising:
a housing less than 1¼ inch in diameter and less than 1¼ inch in length and including an inlet and an outlet and defining a reservoir therein;
a conduit approximately 2 mm in diameter in the housing extending through the reservoir from the inlet, the conduit having a distal open end spaced from the housing outlet;
a hydrophilic filter canister in the shape of a cylindrical body with a centrally extending channel therein which receives the conduit of the housing and including:
an end wall disposed over the distal open end of the conduit, the end wall having gas ports therethrough in fluid communication between the housing outlet and distal open end of the conduit, and
a hydrophilic column extending from the end wall within the conduit and surrounded by the channel for collecting liquid and wicking said liquid into the reservoir; and
a hydrophobic filter disposed between the hydrophilic filter canister and the housing to block liquid from passing through the outlet.

9. A moisture trap comprising:
a housing including an inlet and an outlet and defining a reservoir therein;
a conduit in the housing extending through the reservoir from the inlet, the conduit having a distal open end spaced from the housing outlet;
hydrophilic material received in the housing filling the reservoir; and
hydrophilic material disposed over the distal open end of the conduit with gas ports therethrough in communication between the housing outlet and the distal open end of the conduit.

10. The moisture trap of claim 9 further including a hydrophilic column extending from the hydrophilic material disposed over the distal open end of the conduit and within the column for collecting liquid and wicking said liquid into the reservoir.

11. The moisture trap of claim 9 further including a hydrophobic filter disposed between the hydrophilic material disposed over the distal open end of the conduit and the housing outlet to block liquid from passing through the outlet.

12. The moisture trap of claim 9 in which the hydrophilic material disposed over the distal open end of the conduit and the hydrophilic material received in the housing filling the reservoir is a single integral canister.

13. The moisture trap of claim 12 in which said canister further includes a hydrophilic column received in the conduit.
